(19) United States
(12) Patent Application Publication
(10) Pub. No.: US 2017/0119692 A1
(43) Pub. Date: May 4, 2017

(54) MICROCAPILLARY POLYMER FILMS FOR DRUG DELIVERY

(71) Applicant: Dow Global Technologies LLC, Midland, MI (US)

(72) Inventors: Nicholas S. GRASMAN, Midland, MI (US); Kevin P. O'DONNELL, Midland, MI (US); True L. ROGERS, Midland, MI (US)

(21) Appl. No.: 15/314,230

(22) PCT Filed: Jun. 5, 2015
(86) PCT No.: PCT/US2015/034405

§ 371 Date: Nov. 28, 2016

Related U.S. Application Data
(60) Provisional application No. 62/011,667, filed on Jun. 13, 2014.

(51) Int. Cl. A61K 9/70; A61K 47/32; A61K 31/192; A61K 47/10

(52) U.S. Cl. CPC A61K 9/7007; A61K 47/10; A61K 47/32; A61K 31/192

(57) ABSTRACT

A drug-containing microcapillary film having:

(a) a matrix comprising a polymer having a glass transition temperature less than 190° C.; wherein the matrix has a thickness from 5 to 2000 microns; (b) channels disposed in parallel in the matrix, wherein the channels are separated from each other by at least 1 micron, and wherein total cross-sectional area of the channels is from 5 to 70% of total cross-sectional area of the film; and (c) at least one drug disposed in the matrix and/or in the channels.

This invention relates to a drug-containing microcapillary polymer film useful in controlled delivery of drugs.

Microcapillary polymer films have been described. For example, U.S. Pub. No. 2013/0288016 discloses microcapillary polymer films in which the microcapillaries contain a filler material. However, the prior art does not disclose or suggest a drug-containing microcapillary polymer film useful for controlled delivery of drugs.

### STATEMENT OF INVENTION

The present invention provides a drug-containing microcapillary film comprising: (a) a matrix comprising a polymer having a glass transition temperature less than 190° C.; wherein the matrix has a thickness from 5 to 2000 microns; (b) channels disposed in parallel in said matrix, wherein said channels are separated from each other by at least 1 micron, and wherein total cross-sectional area of the channels is from 5 to 70% of total cross-sectional area of the film; and (c) at least one drug disposed in said matrix, in said channels or a combination thereof.

### DETAILED DESCRIPTION

Percentages are weight percentages (wt %) and temperatures are in ° C., unless specified otherwise. All operations described herein were performed at room temperature (20-25° C.), unless specified otherwise Weight percentages of monomer residues are based on the total weight of monomer residues in the polymer. All polymer Tg and Tm values are determined by differential scanning calorimetry (DSC) according to ASTM D3418.

Preferably, the thermoplastic material comprising the matrix is a polymer accepted for use in pharmaceutical applications, preferably a poly(alkylene oxide) (e.g., poly(ethylene oxide) (including materials designated as polyethylene glycols), poly(propylene oxide), poly(butylene oxide), and mixtures and copolymers thereof), an alkyl (including substituted alkyl) cellulose polymer (e.g., methyl cellulose, ethyl cellulose, hydroxyethyl cellulose, hydroxypropyl cellulose, carboxymethyl cellulose, and mixtures and copolymers thereof, e.g., hydroxypropyl methyl cellulose, hydroxypropyl methylcellulose acetate succinate, hydroxypropyl methylcellulose phthalate, cellulose acetate phthalate), homopolymers and copolymers of N-vinyl lactams and N-Vinyl pyrrolidone (e.g., polyvinylpyrrolidone (PVP), copolymers of N-vinyl pyrrolidone and vinyl acetate or vinyl propionate), polyacrylates and polymethacrylates (e.g., methacrylic acid/ethyl acrylate copolymers, methacrylic acid/methyl methacrylate copolymers, amino methacrylate copolymers), polyvinyl alcohol, and/or ethylene vinylacetate.

Preferably, the weight-average molecular weight (M_{w}) of the polymer is from 1,000 to 10,000,000, preferably from 10,000 to 8,000,000, preferably from 100,000 to 7,000,000.

Preferably, the matrix comprises at least 50 wt % of one or more polymers having a glass transition temperature less than 190° C., preferably at least 60 wt %, preferably at least 70 wt %, preferably at least 80 wt %, preferably at least 90 wt %, preferably at least 95 wt %. Other components of the matrix may include, e.g., the drug and plasticizers.

Drugs are pharmacologically active substances used to treat humans or animals. Preferably, drugs are approved by the relevant regulatory agency for treatment of conditions occurring in humans or animals. Especially preferred drugs include, e.g., antifungals, antibiotics, anti-inflammatory, anti-migraine, antihistamines, analgesics, antioxidants, nicotine, antipsychotics and life-style drugs (e.g. erectile dysfunction). Drugs may be incorporated into the matrix and/or the channels in their pure form or as a mixture with a polymer or other carrier. The polymer may be the same as the one used in the matrix or different. Preferred polymers for a drug/polymer mixture are the same as those preferred for the matrix. Preferred carriers include, e.g., lipids (preferably oils, preferably vegetable oils), salts, cyclodextrins and alcohols (preferably ethyl or isopropyl alcohol). More than one drug may be incorporated into the microcapillary film; drugs may be in the same or different channels, may be divided between the matrix and the channels, or any other combination, and if the drug is in the channel it may be in the liquid or solid state. When the drug(s) is present only in the matrix, the channels may be empty or may be filled with a polymer different from the matrix; preferably the channels are empty. The amount of drug(s) in the matrix and/or the channels may vary greatly depending on the desired dosage of the drug, the desired release profile, the solubility or dispersibility of the drug in a carrier, and other factors known to those in the field of drug delivery. Drugs may be delivered by a variety of mechanisms, e.g., by flow and/or diffusion along the channels, by diffusion through the matrix to the outer surface of the film, by diffusion from the matrix into the channels followed by flow and/or diffusion along the channels, by erosion of the matrix followed by flow and/or diffusion from or along the channels, by pore formation in the matrix followed by flow and/or diffusion from the channels, or by a combination thereof. Flow along the channels may occur in the presence or absence in the channels of an aqueous biological medium from the human or animal being treated; said aqueous biological medium could be present in the channels if the channels were empty and the drug was in the matrix or if a polymer present in the channels draws said medium in. The area and thickness of the film used as a drug delivery system may be varied depending on the desired dosage, desired drug exposure time or drug release time, the means of administration to the human or animal, and other factors known to those in the field of drug delivery systems. The term “controlled delivery,” as used herein encompasses rapid delivery of a drug from the film as well as gradual release of the drug over a longer period of time and also delayed delivery of the drug (i.e., delivery of the drug after a delay from administration of the film, said delay being approximately of the same order of magnitude as the delivery time).

Preferably, the drug is introduced into the channels or the matrix in the form of a drug/carrier composite having a melting point no greater than 230° C., preferably no greater than 180° C., preferably no greater than 160° C. Drugs which are liquid at room temperature may be used, but preferably their boiling points at normal atmospheric pressure are at least 150° C., preferably at least 200° C.

Preparation of films having microcapillary channels has been described, e.g., in U.S. Pat. No. 5,046,936. Even when needles having circular cross sections are used to generate the channels, the cross sections of the channels in the extruded film, perpendicular to the long dimension of the channels, often are not circular, but have irregular shapes. The diameter of a channel is determined by measuring the longest and shortest distances between the walls of the cross section of the channel, where the distances are measured on lines which cross the center of the channel. The center of the channel is defined as the center of mass of a shape having a uniform cross section identical to that of the channel cross section. Preferably, at least three cross sections are analyzed as described and the arithmetic average thereof used as the diameter. Preferably, the diameter of the channels is at least 1 micron, preferably at least 5 microns, preferably at least 10 microns, preferably at least 20 microns, preferably at least 50 microns, preferably at least 100 microns; preferably no more than 1000 microns, preferably no more than 800 microns, preferably no more than 600 microns, preferably no more than 500 microns, preferably no more than 400 microns, preferably no more than 300 microns, preferably no more than 200 microns. Preferably, the channels are separated from each other by at least 2 microns, preferably at least 10 microns, preferably at least 50 microns; preferably no more than 400 microns, preferably no more than 200 microns, preferably no more than 100 microns.

Preferably, the total cross-sectional area of the channels is no more than 65% of total cross-sectional area of the film, preferably no more than 60%, preferably no more than 55%, preferably no more than 50%; preferably at least 10%, preferably at least 15%, preferably at least 20%, preferably at least 25%, preferably at least 30%.

Preferably, the channels extend through at least 50% of the length of the film, measured in the direction of the channels, preferably at least 70%, preferably at least 80%, preferably at least 90%, preferably at least 95%, preferably at least 98%. In one preferred embodiment of the invention, the channels extend through 100% of the film length, i.e., they are open at the ends of the film. In another preferred embodiment, the channels are sealed at the ends so that they extend through no more than 99.9% of the film length, preferably no more than 99.7%, preferably no more than 99.5%, preferably no more than 99%, preferably no more than 98%.

Preferably, the thickness of the matrix is at least 10 microns, preferably at least 30 microns, preferably at least 50 microns, preferably at least 100 microns, preferably at least 200 microns, preferably at least 250 microns; preferably no more than 1700 microns, preferably no more than 1400 microns, preferably no more than 1200 microns, preferably no more than 1000 microns, preferably no more than 900 microns, preferably no more than 800 microns, preferably no more than 700 microns.

Preferably, the length of the film, i.e., the dimension along the film parallel to the channels, is from 0.1 to 30 cm, preferably from 0.5 to 10 cm, preferably from 1 to 3 cm. Preferably, the width of the film is from 0.1 to 30cm, preferably from 0.5 to 10 cm, preferably from 1 to 3 cm.

### EXAMPLES

### Example 1

### POLYOX Film with Empty Microcapillaries

The extrusion line for making air-filled POLYOX microcapillary films consists of a 3.8-cm KILLION single-screw extruder equipped with a gear pump, transfer lines, an elbow, an air line and a microcapillary die. The plant air was supplied by the air line with a flow meter, and was wide open prior to heating the machine to prevent the blockage of microcapillary pins by the backflow of polymer melt. First, the extruder, gear pump, transfer lines and die were heated to the operating temperatures with a sufficient “soak” time. A representative extrusion temperature profile is shown in Table 1. As the polymer pellets passed through the extruder screw the polymer was brought to the molten state. The extruder screw fed the melt to a gear pump which maintained a substantially constant flow of melt towards the microcapillary die. Then, the polymer melt passed over the microcapillary pins and met with streamlines of air flow, which maintained the size and shape of the microcapillary channels. Upon exiting the extrusion die, the extrudate passed over a chill roll. Once the extrudate was quenched, it was taken by a nip roll and wound by a tension winder. The air flow rate was carefully adjusted in such a way that the microcapillaries would not blow out but maintained reasonable microcapillary dimensions. The line speed was controlled by a nip roll in the rollstack.

TABLE 1
Temperature profile of microcapillary extrusion
line for air-filled POLYOX microcapillary films.

| Extruder | | | | Adaptor | Transfer | Screen | Feed | Die |
|---|---|---|---|---|---|---|---|---|
| Zone 1 | Zone 2 | Zone 3 | Zone 4 | Zone | Line | Changer | block | Zone |
| (° C.) | (° C.) | (° C.) | (° C.) | (° C.) | (° C.) | (° C.) | (° C.) | (° C.) |
| 99 | 110 | 120 | 120 | 130 | 130 | 130 | 130 | 130 |

At a line speed of 0.6 m/min, the microcapillaries showed a hexagonal shape. As the line speed increased to 3 m/min, they became an elliptical shape and exhibited a larger deformation. The increase in air flow rate led to a significant increase in the microcapillary size. Additionally, at a lower air flow rate (e.g. 25 ml/min), the smallest film thickness occurred at the locations of the film where microcapillaries resided. The uniformity of film thickness became much better as the air flow rate increased, because the larger air flow rate afforded higher air pressure to resist the perpendicular force exerted on the film section having microcapillaries during the draw-down process.

To investigate the variation of microcapillary size under different processing conditions, the area of microcapillaries was integrated and divided by the film cross-section area, giving the area percentage of microcapillaries in the film. It can be seen from Table 2 that the area percentage of microcapillaries in the film increased with both line speed and air flow rate. The effect of line speed on the area percentage of microcapillaries in the film arose from the thinning of microcapillary walls during draw-down process. At an air flow rate of 25 ml/min, the area percentage of microcapillaries in the film increased from 18.8% to 32.0% as the line speed increased from 0.6 m/min to 3 m/min In contrast, for an air flow rate of 100 ml/min, the area percentage of microcapillaries in the film increased from 26.4% to 52.6% when the line speed increased from 0.6 m/min to 3 m/min

TABLE 2

| | | | Area of |
|---|---|---|---|
| Air Flow | Line | Average Film | Microcapillaries |
| Rate (mL/min) | Speed (m/min) | Thickness (mm) | in the Film (%) |
| 25 | 0.6 | 1.14 | 18.8 |
| 25 | 1.5 | 0.55 | 32.0 |
| 25 | 3.0 | 0.38 | 32.0 |
| 50 | 0.6 | 1.13 | 22.6 |
| 50 | 1.5 | 0.54 | 34.2 |
| 50 | 3.0 | 0.34 | 49.6 |
| 100 | 0.6 | 1.16 | 26.4 |
| 100 | 1.5 | 0.71 | 42.5 |
| 100 | 3.0 | 0.41 | 52.6 |

### Example 2

### Polyolefin-Filled or Drug-Filled POLYOX Microcapillary Films

The line for producing microcapillary coextrusion films consists of a 3.8-cm KILLION single-screw extruder to supply polymer melt for the matrix of microcapillary coextrusion films and a 1.9-cm KILLION single-screw extruder to supply polymer melt for the microcapillaries via a transfer line to the microcapillary die. This microcapillary die with a width of 5.08 cm and a gap of 1.5 mm possesses 42 microcapillary pins. The typical experimental protocol for making microcapillary films was given as follows. First, the extruders, gear pump, transfer lines and die were heated to the operating temperatures with a sufficient “soak” time. Temperature profiles for the 3.8-cm and 1.9-cm KILLION single-screw extruders during microcapillary coextrusion process are shown in Table 3. Polyolefin or POLYOX/ketoprofen (70/30, wt %) blended pellets were charged into the hopper of the 1.9-cm KILLION single-screw extruder, and the screw speed was slowly turned up to the target value. As the drug/polymer melt exited the microcapillary pins, POLYOX pellets were filled into the hopper of the 3.8-cm KILLION single-screw extruder and the main extruder turned on. The extruder screw of the 3.8-cm KILLION single-screw extruder fed the melt to a gear pump which maintained a substantially constant flow of melt towards the microcapillary die. Then, this polymer melt was divided into two streams, which met with polymer strands from the microcapillary pins. Upon exiting the extrusion die, the extrudate was cooled on a chill roll on a rollstack. Once the extrudate was quenched, it was taken by a nip roll and wound by a tension winder. The line speed was controlled by a nip roll in the rollstack.

TABLE 3
Temperature profiles of microcapillary coextrusion line for polyolefin
or drug-filled POLYOX microcapillary coextrusion films.

| | Extruder Zone | | | | Adaptor | Transfer | Screen | Feed | Die |
|---|---|---|---|---|---|---|---|---|---|
| | 1 | 2 | 3 | 4 | Zone | Line | Changer | block | Zone |
| Extruder | (° C.) | (° C.) | (° C.) | (° C.) | (° C.) | (° C.) | (° C.) | (° C.) | (° C.) |
| 3.8-cm | 99 | 110 | 120 | 120 | 130 | 130 | 130 | 130 | 130 |
| 1.9-cm | 110 | 120 | 120 | NA | NA | 120 | NA | NA | NA |

NA = not applicable

TABLE 4
Area percentage of microcapillaries in POLYOX/polyolefin
microcapillary coextrusion films at varying screw speed of
the 1.9-cm Killion extruder and different line speeds.

| | Screw Speed of | | Area of |
|---|---|---|---|
| Line | 1.9-cm KILLION | Average Film | Microcapillaries |
| Speed (ft/min) | extruder (rpm) | Thickness (mm) | in the Film (%) |
| 5 | 10 | 0.61 | 21.3 |
| 5 | 20 | 0.70 | 31.6 |
| 5 | 30 | 0.80 | 41.3 |
| 5 | 40 | 0.86 | 41.5 |
| 10 | 10 | 0.38 | 19.5 |
| 10 | 20 | 0.43 | 30.3 |
| 10 | 30 | 0.45 | 38.5 |
| 10 | 40 | 0.54 | 42.1 |

TABLE 5
Area percentage of microcapillaries in POLYOX/ketoprofen
microcapillary coextrusion films at varying screw speed of
the 1.9-cm KILLION extruder and different line speeds.

| | Screw Speed of | | Area of |
|---|---|---|---|
| Line | 1.9-cm Killion | Average Film | Microcapillaries |
| Speed (ft/min) | extruder (rpm) | Thickness (mm) | in the Film (%) |
| 5 | 10 | 0.60 | 18.5 |
| 5 | 20 | 0.68 | 23.3 |
| 5 | 30 | 0.81 | 28.5 |
| 5 | 40 | 0.67 | 36.1 |
| 10 | 10 | 0.39 | 19.9 |
| 10 | 20 | 0.45 | 22.6 |
| 10 | 30 | 0.36 | 26.6 |
| 10 | 40 | 0.40 | 35.4 |

TABLE 6
Dissolution of ketoprofen from microcapillary films
in 900 mL pH 5.8 phosphate buffer at 37° C.

| | Percent Ketoprofen | Percent Ketoprofen |
|---|---|---|
| Time | Released (20 RPM, | Released (40 RPM, |
| (Minutes) | 10′/Minute) | 10′/Minute) |
| 0 | 0 | 0 |
| 5 | 41 | 86 |
| 10 | 72 | 105 |
| 15 | 80 | 111 |
| 20 | 84 | 112 |
| 25 | 88 | 112 |
| 30 | 91 | 112 |
| 35 | 92 | 112 |
| 40 | 94 | 112 |
| 45 | 95 | 112 |
| 50 | 96 | 112 |
| 55 | 96 | 112 |
| 60 | 97 | 112 |

Samples were cut manually to a size containing approximately 100 mg of ketoprofen. Errors in cutting account for the release percentage results above 100%.

1. A drug-containing microcapillary film comprising:
(a) a matrix comprising a polymer having a glass transition temperature less than 190° C.;
wherein the matrix has a thickness from 5 to 2000 microns; (b) channels disposed in parallel in said matrix, wherein said channels are separated from each other by at least 1 micron, and wherein total cross-sectional area of the channels is from 5 to 70% of total cross-sectional area of the film; and (c) at least one drug disposed in said matrix, in said channels or a combination thereof.

2. The composition of claim 1 in which the drug is introduced into the channels or the matrix in the form of a drug/carrier composite having a melting point no greater than 230° C.

3. The composition of claim 2 in which the thickness of the matrix is from 200 to 1700 microns.

4. The composition of claim 3 in which the channels extend through 50-100% of film length, measured in the direction of the channels.

5. The composition of claim 4 in which the channels have a diameter from 50 to 800 microns.

6. The composition of claim 5 in which the channels are separated from each other by at least 2 micron.

7. The composition of claim 1 in which the polymer is selected from the group consisting of poly(alkylene oxides), cellulose ether and/or cellulose ester polymers, poly(ethylene glycols), polyacrylates, polymethacrylates, homopolymers and copolymers of N-vinyl lactams and N-Vinyl pyrrolidone, and combinations thereof.

8. The composition of claim 7 in which the drug is introduced into the channels or the matrix in the form of a drug/carrier composite having a melting point no greater than 230° C.

9. The composition of claim 8 in which the thickness of the matrix is from 200 to 1700 microns.

10. The composition of claim 9 in which the channels extend through 50-100% of film length, measured in the direction of the channels.
